(19) United States
(12) Patent Application Publication
(10) Pub. No.: US 2007/0032795 A1
(43) Pub. Date: Feb. 8, 2007

(54) Stereotactic upper body fixation and positioning device

(76) Inventors: Adam Schloesser, Mount Horeb, WI (US); Jesse Darley, Madison, WI (US); John Hughes, Madison, WI (US); Gustavo Olivera, Madison, WI (US); Eric Schloesser, Mount Horeb, WI (US)

Correspondence Address: MICHAEL BEST & FRIEDRICH, LLP, 100 E WISCONSIN AVENUE, MILWAUKEE, WI, 53202, US

(21) Appl. No.: 10/567,480

(22) PCT Filed: Aug. 9, 2004
(86) PCT No.: PCT/US04/25807

§ 371 Date: Oct. 17, 2006

Related U.S. Application Data
(60) Provisional application No. 60/493,596, filed on Aug. 8, 2003.

(51) Int. Cl. A61B 19/00

(52) U.S. Cl. 606/130

(57) ABSTRACT

The present invention provides a positioning device for radiation therapy equipment. In particularly, a stereotactic upper body fixation and positioning device for use in conjunction with a patient positioning couch and delivery system of a radiation therapy machine to provide precise, verifiable, and adjustable positioning in the three rotational axes of movement referred to as roll, pitch and yaw.

### CROSS-REFERENCE TO RELATED APPLICATIONS

This application is based on and claims the benefit of U.S. Provisional Application No. 60/493,596, filed on Aug. 8, 2003, and incorporated herein by reference.

### BACKGROUND OF THE INVENTION

The present invention relates generally to a positioning device for radiation therapy equipment, and more particularly to a stereotactic upper body fixation and positioning device for use in conjunction with a patient positioning couch and delivery system of a radiation therapy machine to provide precise, verifiable, and adjustable positioning in the three rotational axes of movement referred to as roll, pitch and yaw.

Medical equipment for radiation therapy treats tumorous tissue with high energy radiation. The dose and the placement of the dose must be accurately controlled to ensure both that the tumor receives the prescribed dose, and that damage to the surrounding and adjacent non-tumorous tissue is minimized.

Internal-source radiation therapy places capsules of radioactive material inside the patient in proximity to the tumorous tissue. Dose and placement are accurately controlled by the physical positioning of the isotope. However, internal-source radiation therapy has the disadvantages of any surgically invasive procedure, including discomfort to the patient and risk of infection.

External-source radiation therapy uses a radiation source that is external to the patient, typically either a radioisotope, such as Cobalt-60, or a high energy x-ray source, such as a linear accelerator. The external source produces a collimated beam directed into the patient to the tumor site. External-source radiation therapy avoids some of the problems of internal-source radiation therapy, but it undesirably and necessarily irradiates a significant volume of non-tumorous or healthy tissue in the path of the radiation beam along with the tumorous tissue.

The adverse effect of irradiating of healthy tissue may be reduced, while maintaining a given dose of radiation in the tumorous tissue, by projecting the external radiation beam into the patient at a variety of “gantry” angles with the beams converging on the tumor site. The particular volume elements of healthy tissue, along the path of the radiation beam, change, reducing the total dose to each such element of healthy tissue during the entire treatment.

The irradiation of healthy tissue also may be reduced by tightly collimating the radiation beam to the general cross-section of the tumor taken perpendicular to the axis of the radiation beam. Numerous systems exist for producing such a circumferential collimation, some of which use multiple sliding shutters which, piecewise, may generate a radio-opaque mask of arbitrary outline.

As part of collimating the beam to the outline of the tumors, the offset angle of the radiation beam, with respect to a radius line between the radiation source and the center of rotation of the radiation source, may be adjusted to allow the treated area to be other than at the center of rotation. Simultaneously changing the offset angle and the width of the radiation beam as a function of gantry angle allows tumorous tissue having an irregular cross-section within a plane parallel to the radiation beam to be accurately targeted. The width and offset angle of the radiation beam may be controlled by the use of a multiple-leaf collimator.

A typical patient stereotactic apparatus has involved a head ring which is attached by posts and sharpened head screws to a patient's head. The ring encircles the patient's head, and is secured to the head by posts or head screws that can be securely placed into the outer table of the patient's skull. Alternately, the ring may have a dental impression to register to the patient's teeth, or the ring may have a mask mold of the face or head to immobilize relative to the head's external contours. The ring can accept graphic reference localizers which enable scanner index data from tomographic scanning such as CT, MRI, PET, etc. to be used to relate two-dimensional or three-dimensional tomographic scan data from an image scanner to the coordinate reference frame of the ring. Once the “stereotactic coordinates” of a selected target position seen in the image scan data are determined relative to ring, a stereotactic arc system can be used attached to the ring to direct a probe to the physical target corresponding to the selected target position seen from these scan image data. In another embodiment, the ring can be attached to a couch or table of an imaging or radiation therapy machine by means of ring holders, and a delivery of radiation from a LINAC through a collimator system can be directed to the stereotactic target coordinates of the selected target position. These are examples of stereotaxy using a patient immobilizer and graphic reference means from the prior art.

The use of skull-attached head rings, dental impression or mask impression attached head rings have limited usefulness for irradiation of the head region. Specifically, the ring is an obstruction to probe and also to radiation beams, for which it may be desirable to aim at targets at the very lowest part of the skull, the nasal pharynx, jaw, neck, and upper thorax. In this situation, a ring structure placed around the head is obstructive and an impediment to desired beam entry directions. The ring can be placed high on the patient's head, but this too may limit the solid angle of access of the beams to the skull base and nasopharynx. The head ring also may prevent wide angulation of the head relative to the couch or patient table in some cases. There are not a lot of head positioning or fixation devices that are radio transparent.

Other examples of prior art head and neck immobilizers include mouth bite type head stabilizers or masks. However, these types of systems have no use of graphic reference means and/or target localizers. They also have no biasing means that can effectively stabilize the head movements. They have no repeat positioning to different couches, and are not obstruction-free.

There are other devices that position and/or restrain the head and neck that allow one degree of movement, but none that offer more than two degrees of movement or rotational freedom.

Therefore, there is a need for a compact, light weight, easy to install and remove from the couch top, and radio transparent fixation and positioning device that can allow adjustment of a patient's position in the three rotational axes, including roll, pitch and yaw.

### SUMMARY OF THE INVENTION

The present invention is a device for accurate fixation positioning and localizing a patient's upper body for radio imaging, radio therapy, and radio surgery procedures. Radio surgery is a term for high precision radiation therapy, especially in the head and neck region. The purpose of the device is to fix a patent on a couch top of a tomotherapy machine, particularly the upper body of the patient on the couch top in a particular position, scan the patient to verify the location of that patient's anatomy, and then dynamically make adjustments to the desired treatment position. These adjustments are made in particular by a control device, interfaced with a feedback device for imaging and therapy applications. The device is used for the purpose of radio therapy or radio surgery but not necessarily on the delivery device, it could be used on the diagnostic portion of the device as well. The device will preferably have adjustment resolution quality assurance mechanisms in place to ensure structural and positional accuracy.

The present invention provides a standard adaptable, head fixation, positioning, and adjustable device for precise, repeatable, verifiable localization of a patient's head, neck and upper torso for the purpose of radio imaging, radio therapy, and radio surgery procedures. The device may not necessarily be used on the delivery device of the tomotherapy machine, but could be used on the diagnostic portion of tomotherapy machine. The device of the present invention may also be adaptable for use on CT, MRI, PET, or any other radiotherapy imaging machines.

Applications for the device of the present invention include fractionated, stereotactic radio therapy for the head, brain and neck; stereotactic radio surgery for the head, brain and neck; and stereotactic imaging for the head, brain and neck. The current design includes provisions for fixating the head, neck and upper torso of the patient.

The present invention is a medical device for use in conjunction with a patient positioning table and existing upper body immobilization systems. Provides precise and variable positioning in the three rotational degrees of movement, pitch, roll and yaw.

The present invention is an adaptable head and neck fixation and positioning device for precise repeatable verifiable localization of a patient's head and neck region for the purpose of radio imaging, radio therapy and radio surgery. Applications include fractionated, stereotactic radio therapy for the head and neck, stereotactic radio surgery for the head and neck, and stereotactic imaging for the head and neck. For use in high precision radiation therapy in the head and neck region.

The present invention is a stereotactic upper body fixation and positioning device for use in conjunction with a patient positioning couch and delivery system of a radiation therapy machine to provide precise, verifiable, and adjustable positioning in the three rotational axes of movement referred to as roll, pitch and yaw

The purpose of the device is to fix a patient on a couch, particularly the head and neck of the patient on the couch in a particular position, to scan the patient and verify the location of that patient's anatomy following a registration image, and then make an adjustment, if necessary, such that the position of the patient corresponds to the desired treatment position.

The device is able to move into any position within a specified range and hold that position.

The device of the present invention is very compact, light weight, and easy to install and remove from the couch top. These are advantages over the prior art because most positioning devices that work well are actually large elaborate cumbersome devices that are mounted to the floor.

Various other features, objects, and advantages of the invention will be made apparent to those skilled in the art from the following detailed description.

### BRIEF DESCRIPTION OF THE DRAWINGS

FIG. 1 is a perspective, cut-away view of a radiation therapy system providing for the acquisition of radiographic projections and for the generation of high energy radiation therapy beams and showing a patient couch for supporting a patient thereon;

FIG. 2 is a front perspective view of an embodiment of a stereotactic upper body fixation and positioning device in accordance with present invention;

FIG. 3 is a side plan view of the device of FIG. 2 with the device in a centered and level position;

FIG. 4 is a side plan view of the device of FIG. 2 with the device in a centered and raised position;

FIG. 5 is a rear plan view of the device as shown in FIG. 3;

FIG. 6 is a rear plan view of the device as shown in FIG. 4;

FIG. 7 is a rear plan view of the device of FIG. 2 with the device in a centered and tilted right position;

FIG. 8 is a rear plan view of the device of FIG. 2 with the device in a centered and tilted left position;

FIG. 9 is a rear perspective view of the device of FIG. 2 with the device track in a right position and the device in a raised position;

FIG. 10 is a rear perspective view of the device of FIG. 2 with the device track in the right position and the device in a tilted left position;

FIG. 11 is a rear perspective view of the device of FIG. 2 with the device track in a left position and the device in a raised position; and

FIG. 12 is a rear perspective view of the device of FIG. 2 with the device track in the left position and the device in a tilted right position.

### DETAILED DESCRIPTION OF THE INVENTION

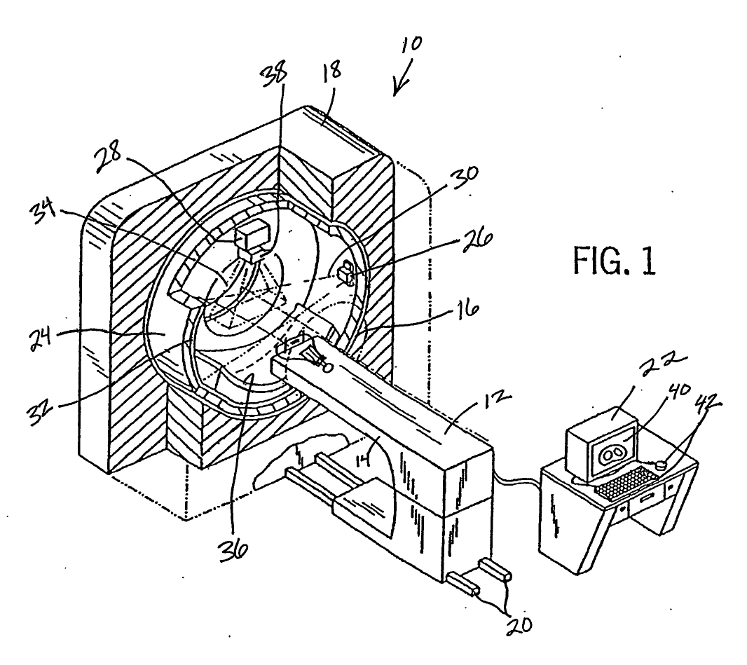

Referring now to the drawings, FIG. 1 illustrates a radiation therapy machine 10 suitable for use with the present invention. The radiation therapy machine 10 preferably includes a radiotranslucent couch 12 having a cantilevered top 14. The couch top 14 is received within a bore 16 of an annular housing 18 of the radiation therapy machine 10 with movement of the couch 12 along tracks 20 extending along a longitudinal axis translation as described by the IEC 61217 “T” coordinate system The couch 12 is preferably disposed along the longitudinal axis and may slide along that axis through the bore 16 passing first the front surface and then the rear surface. The couch 12 is supported along guide tracks 20 and moved by a motorized drive, such as is well known in the art, so that its position may be controlled by a computer 22 as will be described.

The couch 12 also includes an internal track assembly and elevator (not shown) to allow adjustment of the couch top 14 in a lateral axis translation and a vertical axis translation as described by the IEC 61217 “T” coordinate system. Motion in the lateral and vertical directions are limited by the diameter of the bore 16.

A rotating gantry 24, coaxial with the bore 16 and positioned within the housing 18, supports an x-ray source 26 and a high energy radiation source 28 on its inner surface. The x-ray source 26 may be a conventional rotating anode x-ray tube, while the radiation source 28 may be any source of treatment radiation including one producing x-rays, accelerated electrons, protons or heavy ions such as are understood in the art. The x-ray source 26 and a radiation source 28 rotate with the gantry 24 about a center of rotation near the top 14 of patient couch 12 when the couch top 14 is positioned within the bore 16.

The x-ray source 26 is collimated to produce a fan beam 30 crossing the bore 16 and thus the couch top 14 when the couch top 14 is positioned with the bore 16. The fan beam 30 diverges about a central axis whose angle is controlled by the position of the gantry 24. The central axis will henceforth be termed the projection axis.

After exiting the couch top 14, the fan beam 30 is received by a linear array detector 32 positioned diametrically across from the x-ray source 26. Thus, the rotating gantry 24 permits fan beam radiographic projections of a patient on the couch top 14 to be acquired at a variety of angles about the patient.

The radiation source 28 is mounted so as to project a fan beam of high energy radiation 34, similar to the fan beam 30, but crossing the fan beam 30 at right angles so as to be received on the other side of the gantry 24 by radiation detector and stop 36. In an alternative embodiment, the stop 36 is replaced by a detector to provide an alternative to the detector for deducing motion of the patient. The fan beam of high energy radiation 34 diverges about a radiation axis centered within the beam and perpendicular to the projection axis.

The radiation source 28 has a collimator 38 mounted in front of it to divide the beam of high energy radiation 34 into multiple adjacent rays whose energy and/or fluence may be individually controlled. As used herein, control of the energy and/or fluence of the rays should be understood to include not only the energy of individual x-ray photons (or particles in the case of radiation therapy using electrons, protons or heavy ions) but alternatively or in addition the total number of photons or particles such as is a function of fluence, fluence rate and exposure time. In the case of radiotherapy using particles, the energy of the particles, fluence and fluence rate may be controlled using sinograms which may be modified by the present invention as will be apparent from the following description.

A collimator suitable for fluence control is preferred. A simple modification of this collimator using wedge filters may be used for particle energy control. Alternatively, a scanning single beam system may be used or other system providing a set of individually modulated rays. The location of the radiation source and x-ray source are precisely characterized so that images obtained from the radiation source may be used to aim the radiation source.

The computer 22 preferably includes a display screen 40 and input devices 42, such as a user entry mouse and keyboard that are well known in the art, and connected to the radiation therapy machine 10 to control motion of the couch 12 and to coordinate operation of the gantry 24 together with the radiation source 28 and x-ray source 26 and to collect data from the linear array detector 32 during a scan of a patient according to methods well known in the art.

The stereotactic upper body fixation and positioning device 50 of the present invention is preferably mounted on the couch top 14, or any other add-on to the couch 12, the couch top 14 having three ranges of motion including, longitudinal, lateral and vertical motion. The couch 12 can be vertically adjusted, laterally adjusted and horizontally adjusted to accurately position the patient within the radiation therapy machine 10. The stereotactic upper body fixation and positioning device 50 of the present invention provides an additional three ranges of motion of a patient's head, neck and upper torso that are defined as roll, pitch and yaw. Together with the three ranges of motion of the couch 12, the present invention provides six ranges of motion. The position of the stereotactic upper body fixation and positioning device 50 is adjustable and may be locked into place for radiation treatment of a patient. These adjustments and translations are inherent to the present invention. The complex motions of the present invention described above are achieved by combining the couch 12 and the stereotactic upper body fixation and positioning device 50 motions.

The rotational movements of the stereotactic upper body fixation and positioning device 50 are referred to as roll, pitch, and yaw.

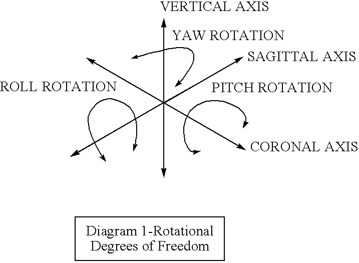

Roll is one of the three rotational axes commonly used to describe the rotation of an object about the sagittal axis of a person's body. A person lying flat on their back, near parallel to a horizontal plane, who rotates their head about their neck axis left or right in a range of −90 to +90 degrees conveys the rotation of “roll” as shown in Diagram 1 above.

Pitch is one of the three rotational axes commonly used to describe the rotation of an object about the coronal axis of a person's body. A person lying flat on their back, near parallel to a horizontal plane, who lifts their head straight in an arched motion as their chin arcs toward their neck is conveying the rotation of “pitch” as shown in Diagram 1 above.

Yaw is one of the three rotational axes commonly used to describe the rotation of an object about the perpendicular intersection of the sagittal and coronal planes referred to as the vertical axis. A person lying flat on their back, near parallel to a horizontal plane, who translates their head directly from one shoulder to the other conveys the rotation of “yaw” as shown in Diagram 1 above.

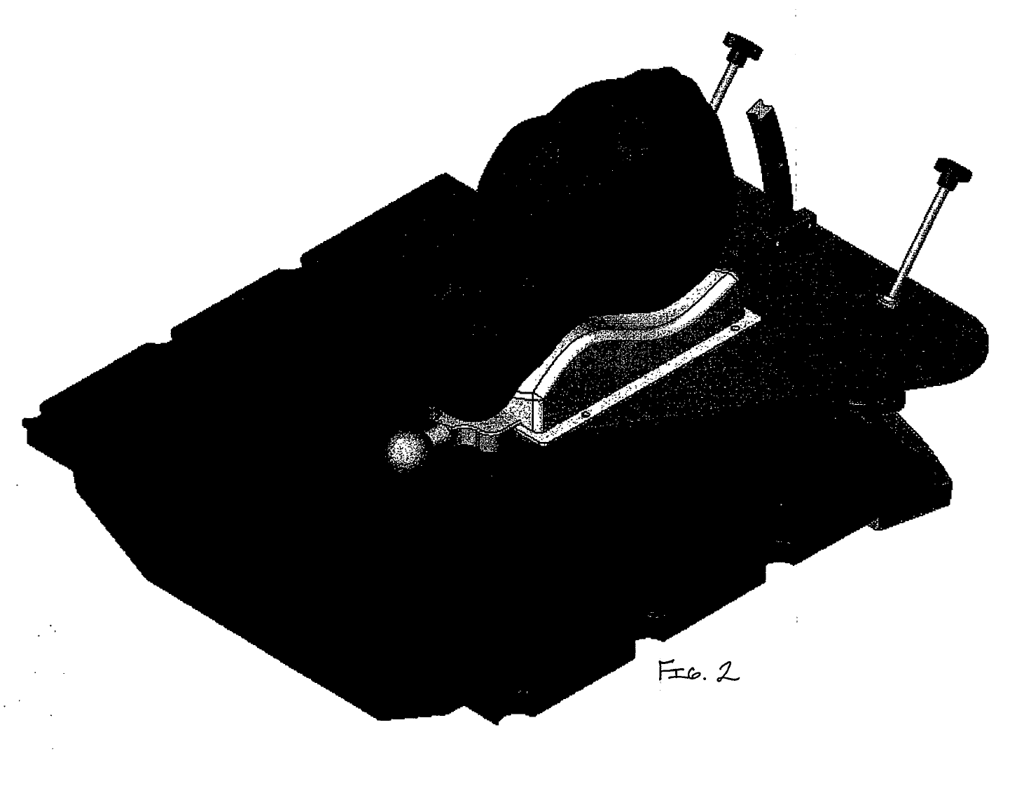

FIG. 2 illustrates a perspective view of an embodiment of a stereotactic upper abdominal fixation and positioning device 50 of the present invention. The stereotactic upper abdominal fixation and positioning device 50 is preferably used for accurately positioning and localizing a patient's head, neck and upper lumbar regions for radio imaging, radio therapy and radio surgery procedures.

The stereotactic upper abdominal fixation and positioning device 50 is preferably mounted to one end of the couch 12 and includes a plate member 52 having a pivoting member 54 attached to one end 56 of the plate member 52 by a bracket 58, a vertically oriented bearing assembly 60 having a vertically oriented curved linear bearing 62 extending through an opening 64 in the opposite end 66 of the plate member 52, a horizontally oriented bearing assembly 68 attached to the end 70 of the couch 12 toward the bottom of the couch 12, and at least two adjustable pivoting rod assemblies 72 attached to the vertically oriented and horizontally oriented bearing assemblies 60, 68. The stereotactic upper abdominal fixation and positioning device 50 also preferably includes a support member 74 attached to the top surface of the plate member 52 and the bracket 58 for supporting a patient's head, neck, and upper torso 76 on the plate member 52.

The pivoting member 54 is the focal and pivot point of the stereotactic upper abdominal fixation and positioning device 50. The pivoting member 54 preferably touches and pivots on the couch top 14 allowing the stereotactic upper abdominal fixation and positioning device 50 to have roll, pitch and yaw adjustable movements. The pivoting member 54 is preferably the focal point of the vertically oriented and horizontally oriented bearing assemblies 60, 68 and is preferably located between the pelvic and lumbar regions of a patient's body when the patient is laying on the couch 12. The bracket 58 preferably attaches the pivoting member 54 to the plate member 52 and preferably assists in fixating the support member 74 to the plate member 52.

The vertically and horizontally oriented bearing assemblies 60 and 68 and the adjustable pivoting rod assemblies 72 are preferably located on the end of the couch 12 out of the radiation treatment beam. The vertically oriented bearing assembly 60 preferably includes a vertically oriented curved linear bearing 62 having an arc with the focal point of the pivoting member 54 and extending through an opening 64 in the opposite end 66 of the plate member 52 and a movable car 78 that travels along the arc of the curved linear bearing 62. Likewise, the horizontally oriented bearing assembly 68 includes a horizontally oriented curved linear bearing 80 having an arc with the focal point of the pivoting member 54 and a movable car 82 that travels along the arc of the curved linear bearing 80.

Using a series of rack and pinion, compact gear box setups, curved bearing rails and cars, cams and rollers, and bearings, direct and accurate translation in roll, pitch, and yaw will be achieved. Once position has been achieved, a locking mechanism on the cars will hold the position of the cars on the bearings securely. The device may be manually or electronically driven in all three rotational degrees of movement. The mounting “car” provides for highly accurate and direct rotational translation. Movement is preferably by rack and pinion movement with cars on a bearing track that may be locked into position by a locking mechanism. One example of a locking mechanism is a clutch plate locking mechanism with a quick release cam lever similar to those found on bicycle seat post height adjustments.

The adjustable pivoting rod assemblies 72 attached to the vertically oriented and horizontally oriented bearing and track assemblies 60, 68 at least two rods extending through openings in the plate member at the same end thereof as the opening for the curved vertical track

The device is adjustable in the three rotational degrees of freedom, roll, pitch and yaw, and preferably pivots on the pivoting member. Axes of rotation of the device intersect at the single pivot point, the pivot point preferably being the tangent between the pivot member and the top surface of the couch.

The device preferably has adjustment resolution quality assurance mechanisms in place to ensure structural and positional accuracy. As a fundamental safety feature, the device's maximum mechanical limits will be set not to exceed nominal range of joint motion from the lower lumbar region to thoraco-sternum/neck area for men and women body types found in the current 1% to 99th percentile category found in up to date anthropometric data.

The device will predominantly be made out of high-strength materials with low absorption of x-ray radiation. Low-density materials such as carbon fiber, nylon, and acrylic will be used to construct the treatment support member to support and provide restraint provision for patient's upper body. This support member will be fixed and adjusted by means of precision equipment assembled outside of the primary radiation beam. Protective covers and flexible bellows will protect equipment, add additional safety, and provide overall design theme continuity with existing tomotherapy equipment.

The standard available concepts in use for restraining a patient in a fixed position could be interfaced with the device of the present invention. The device is very compact, lightweight and easy to assemble. The device will preferably be less than 50 lbs. in total assembled weight.

The present invention includes curve linear bearings having a fixed arc and fixed radius, with a single interface point. The three axes converge at this single interface or pivot point. The device is preferably made out of a high transmission material, low density materials, such as carbon fiber, nylon, and acrylic.

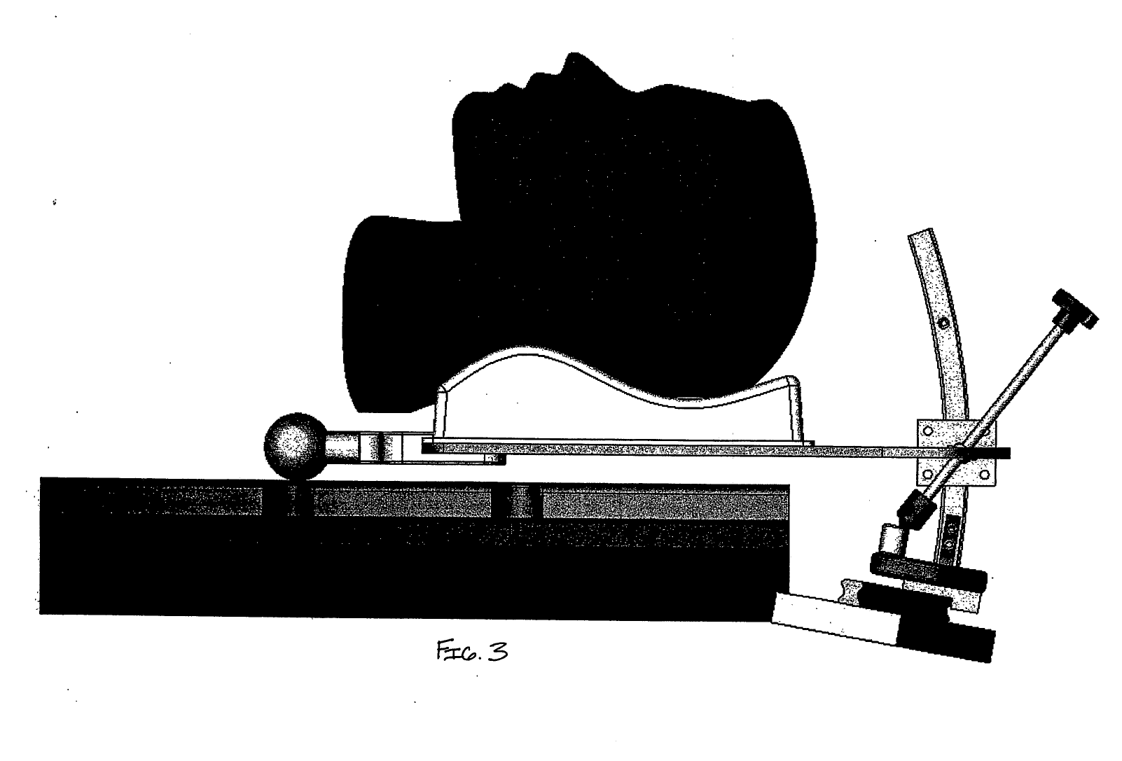

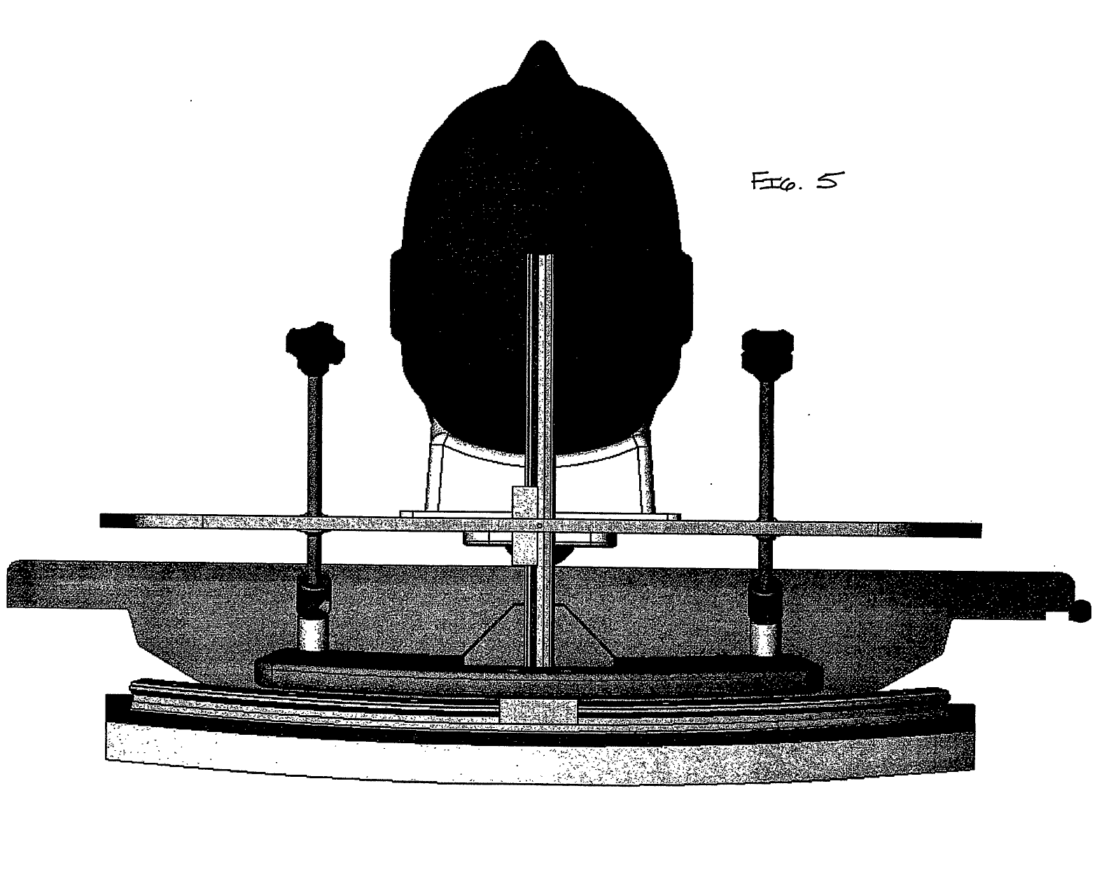

FIG. 3 illustrates the device of the present invention with the plate member and support member attached to the plate member in a centered and level position, parallel with the top of the couch. FIG. 5 is a rear view of the device as shown in FIG. 3.

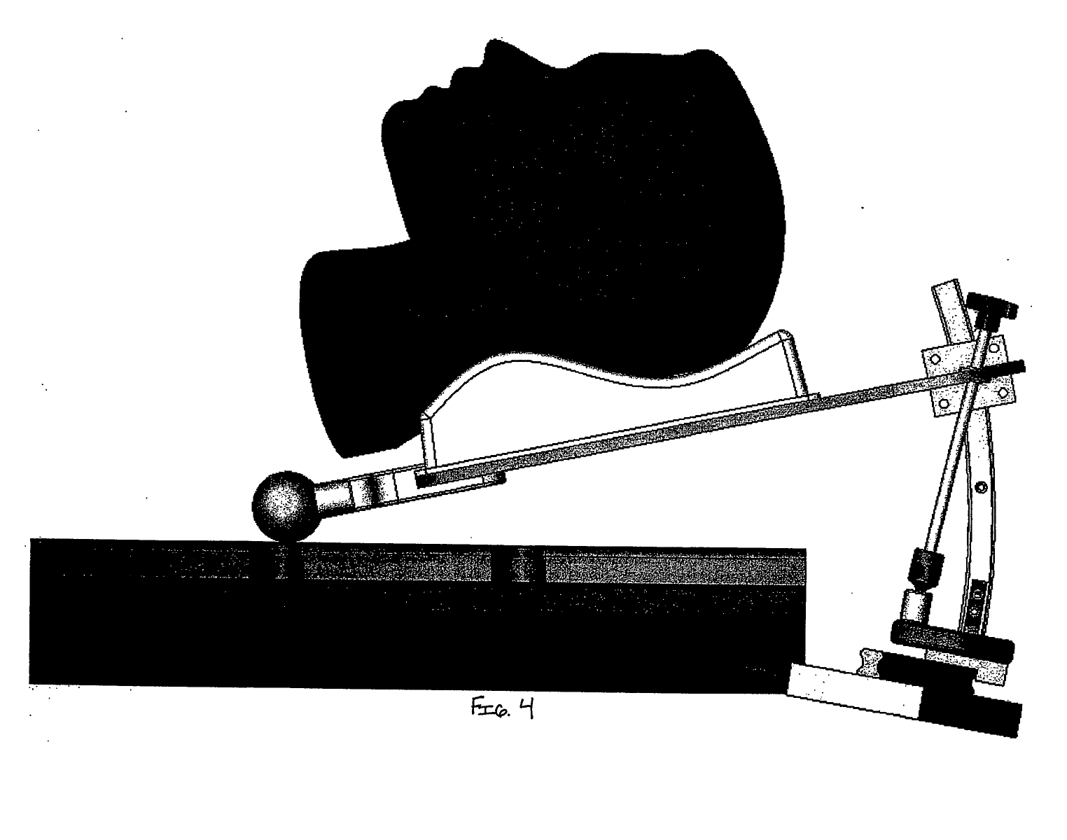

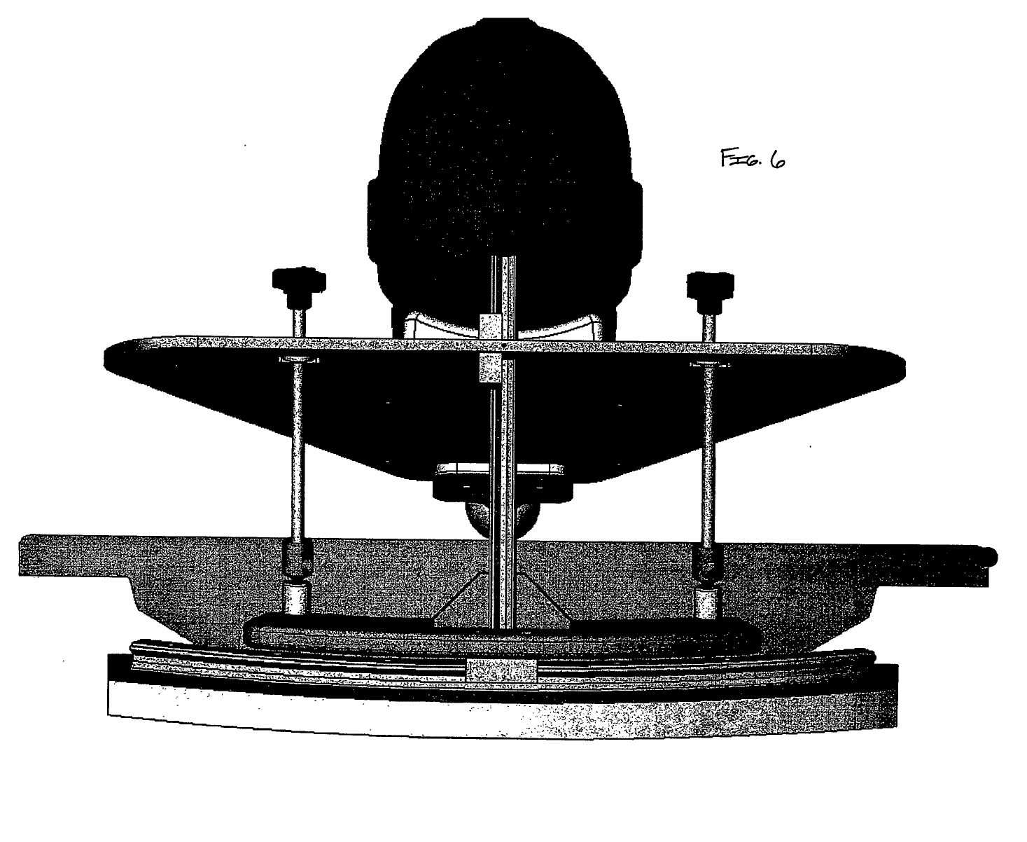

FIG. 4 illustrates the device of the present invention with the plate member and support member in a centered and raised position. This is accomplished by raising the plate member along the curved bearing track and locking it in the desired position with the looking mechanism. FIG. 6 is a rear view of the device as shown in FIG. 4.

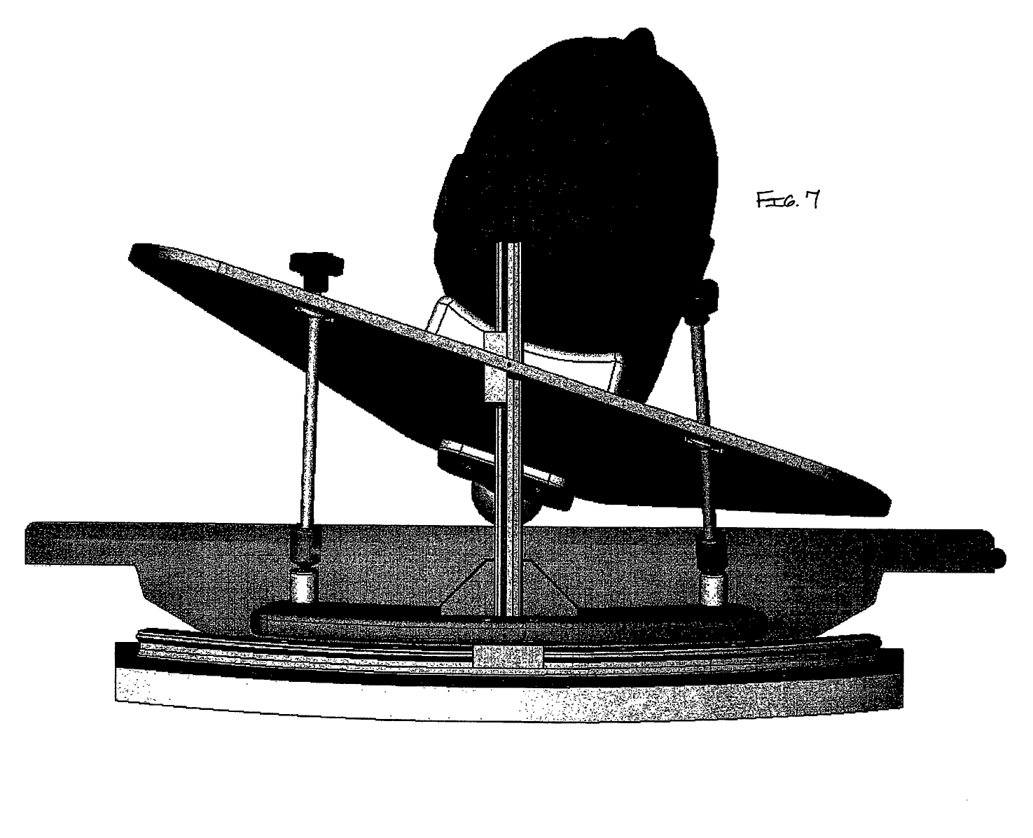

FIG. 7 illustrates the device of the present invention with plate member and support member centered and tilted to the right.

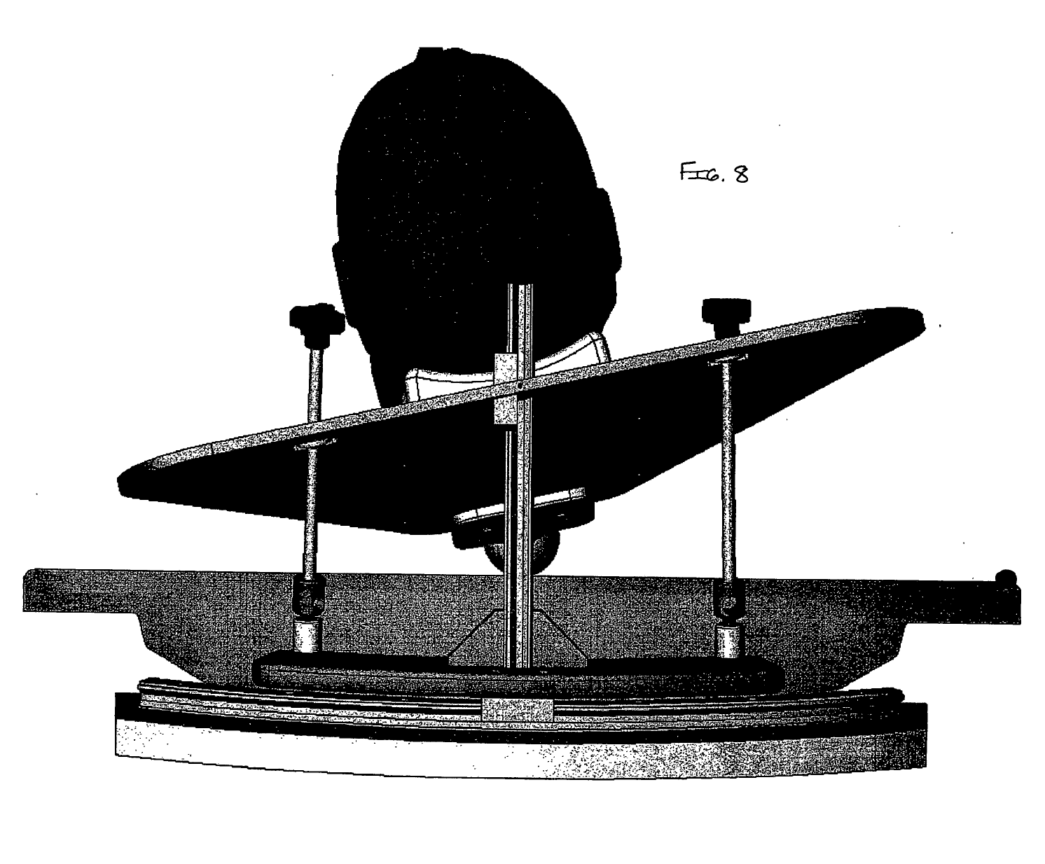

FIG. 8 illustrates the device of the present invention with plate member and support member centered and tilted to the left.

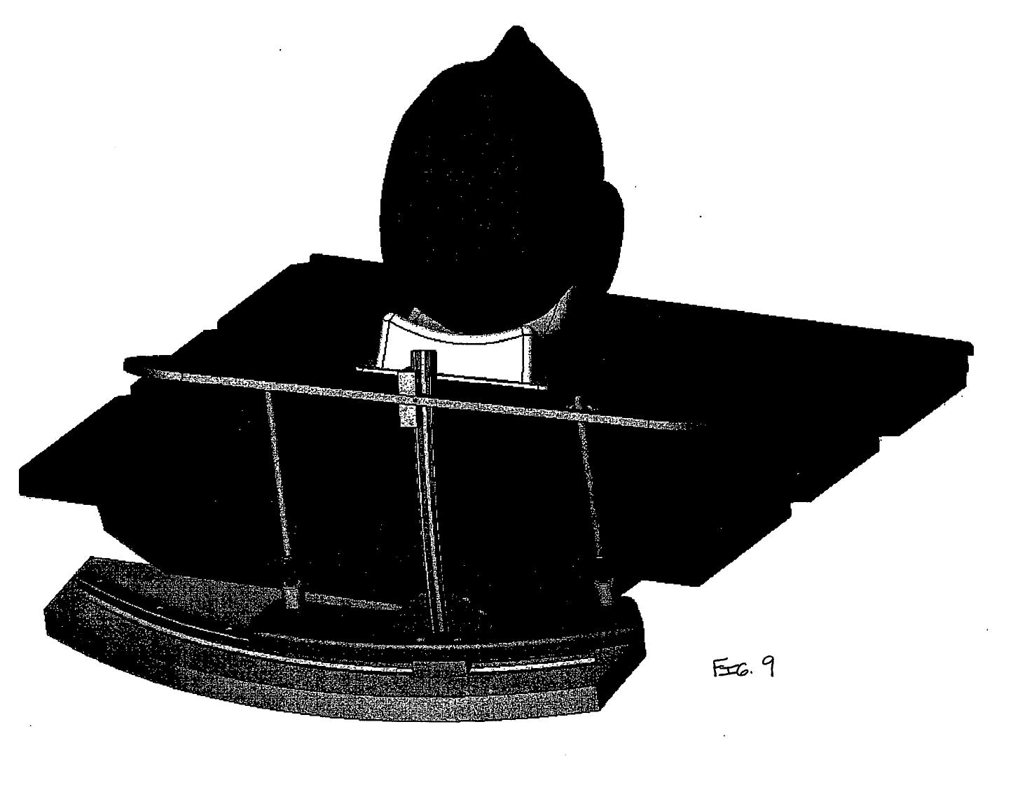

FIG. 9 illustrates the device of the present invention with the device track in a right position and the device in a raised position.

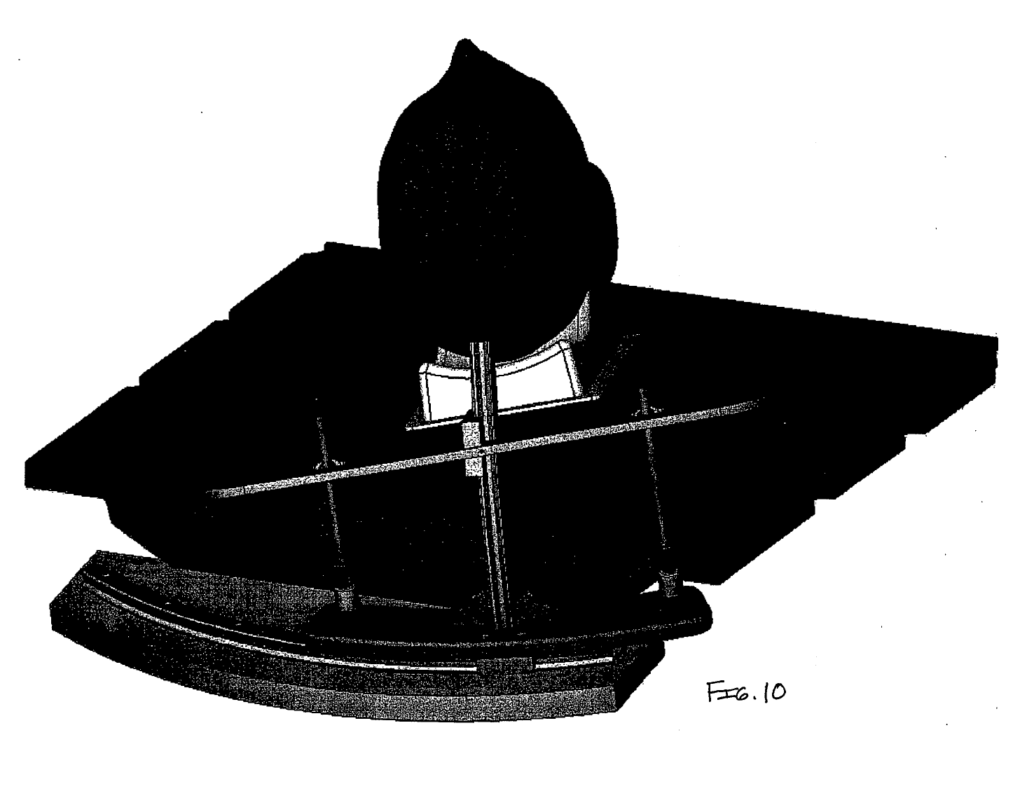

FIG. 10 illustrates the device of the present invention with the device track in the right position and the device in a tilted left position.

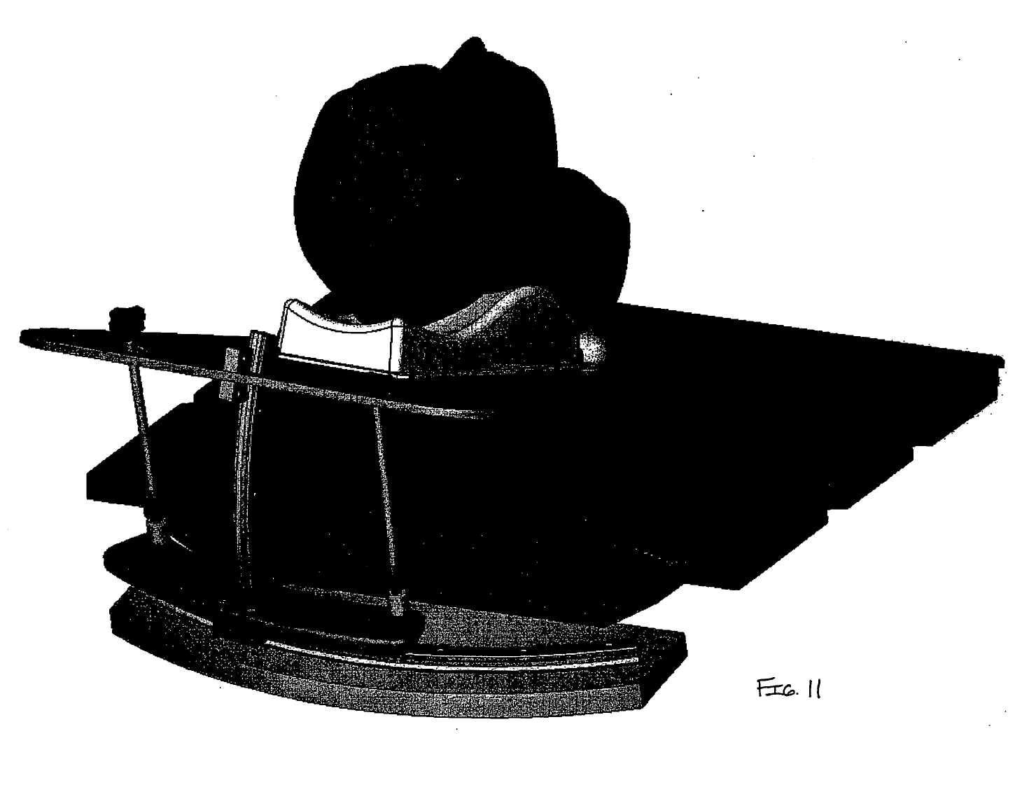

FIG. 11 illustrates the device of the present invention with the device track in a left position and the device in a raised position.

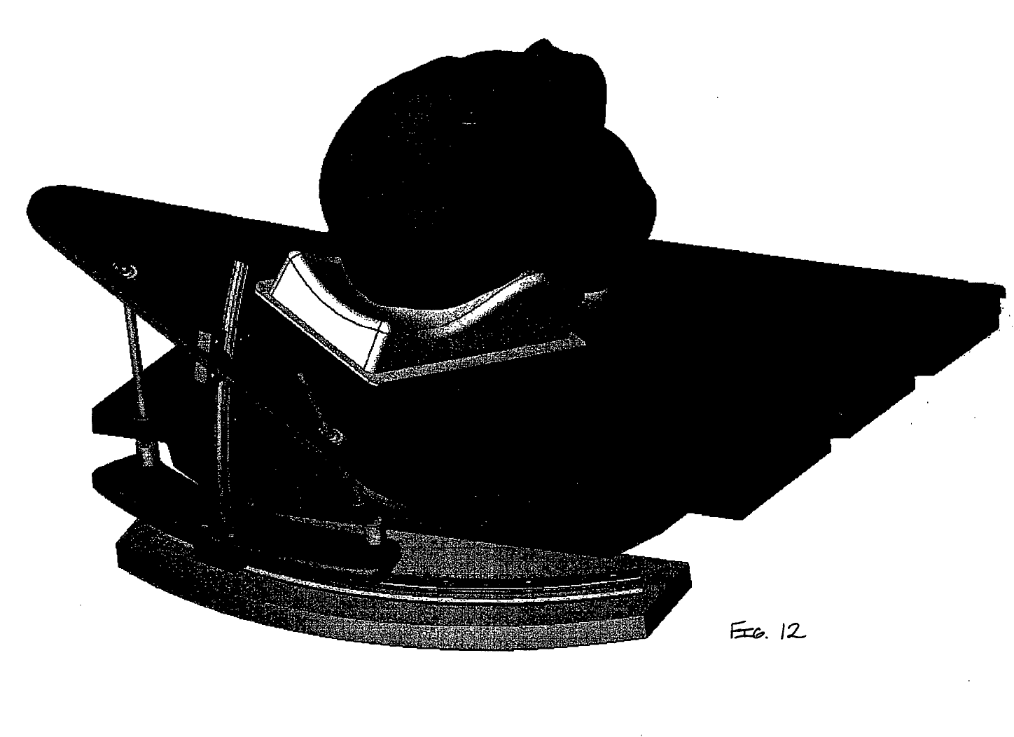

FIG. 12 illustrates the device of the present invention with the device track in the left position and the device in a tilted right position.

In operation, the device provides the ability to move the head and neck into any position within a specified range and precisely hold that position. The device has an accurate tolerance for position fixation. In other words, the device preferably moves accurately and precisely within a specified range.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as recited in the following claims.

1. (canceled)

2. An upper body positioning device connectable to a couch of a radiation therapy treatment system, the upper body positioning device comprising:
a frame;
a projection extending from one end of the frame, the projection defining a pivot point;
a first assembly connectable to a second end of the frame, the first assembly operable to provide a first range of motion of the frame relative to the couch about the pivot point; and
a second assembly connectable to the first assembly, the second assembly operable to provide a second range of motion of the frame relative to the couch about the pivot point.

3. The upper body positioning device of claim 2 wherein the projection is generally spherical-shaped.

4. The upper body positioning device of claim 2 wherein the projection is generally semi-spherical-shaped.

5. The upper body positioning device of claim 2 wherein the first range of motion comprises one of a pitch rotation, a roll rotation, and a yaw rotation and the second range of motion comprises one of a pitch rotation, a roll rotation, and a yaw rotation.

6. The upper body positioning device of claim 2 wherein the first range of motion is different than the second range of motion.

7. The upper body positioning device of claim 2 wherein the first assembly comprises a track and a carriage connected to the frame, the carriage adapted to move along the track.

8. The upper body positioning device of claim 7 wherein the track is oriented in a generally vertical plane, the track being arcuately-shaped, the first assembly providing a pitch movement of the frame relative to the couch as the carriage moves along the arcuately-shaped track in a generally vertical direction.

9. The upper body positioning device of claim 2 wherein the second assembly comprises a track and a carriage adapted to move along the track.

10. The upper body positioning device of claim 9 wherein the track is oriented in a generally horizontal plane, the track being arcuately-shaped, the second assembly providing yaw movement of the frame relative to the couch as the carriage moves along the arcuately-shaped track in a generally horizontal direction.

11. The upper body positioning device of claim 2 further comprising a third assembly connectable to the second end of the frame, the third assembly operable to provide a third range of motion of the frame relative to the couch about the pivot point.

12. The upper body positioning device of claim 11 wherein the third range of motion comprises one of a pitch rotation, a roll rotation, and a yaw rotation.

13. The upper body positioning device of claim 11 wherein the third range of motion is different than the first range of motion.

14. The upper body positioning device of claim 11 wherein the second assembly comprises a track and a carriage adapted to move in a generally horizontal direction along the track, and wherein the third assembly comprises a shaft having a first end connectable to the frame and a second end connectable to carriage of the second assembly, the second end of the shaft adapted to be pivotable with respect to the carriage of the second assembly.

15. The upper body positioning device of claim 14 wherein the third assembly provides roll movement of the frame relative to the couch as the second end of the shaft pivots with respect to the carriage of the second assembly.

16. A positioning device comprising:
a frame adapted to support a body part, the frame having a first axis;
a first assembly adapted to move the body part about the first axis;
a second assembly adapted to move the body part about a second axis oriented perpendicular with respect to the first axis; and
a third assembly adapted to move the body part about a third axis oriented perpendicular with respect to the first axis and the second axis.

17. The positioning device of claim 16 wherein the first axis, the second axis, and the third axis intersect at a common point.

18. The positioning device of claim 16 further comprising a component extending from one end of the frame, the component defining a pivot point, and wherein the first assembly is operable to provide pitch movement of the body part about the pivot point, and wherein the second assembly is operable to provide yaw movement of the body part about the pivot point, and wherein the third assembly is operable to provide roll movement of the body part about the pivot point.

19. A positioning device connectable to a couch of a radiation therapy treatment system, the positioning device comprising:
a frame; and
a component extending from the frame, the component defining a pivot point, the component adapted to provide nearly simultaneously pitch movement, yaw movement, and roll movement of the frame relative to the couch about the pivot point.

20. The positioning device of claim 19 further comprising a first assembly connectable to the frame and adapted to provide the pitch movement of the frame relative to the couch about the pivot point, and further comprising a second assembly connectable to the first assembly and adapted to provide the yaw movement of the frame relative to the couch about the pivot point, and further comprising a third assembly connectable to the first assembly and the second assembly and adapted to provide the roll movement of the frame relative to the couch about the pivot point.

21. The positioning device of claim 19 wherein the component is generally spherical-shaped.
